(19) United States
(12) Patent Application Publication
(10) Pub. No.: US 2007/0299479 A1
(43) Pub. Date: Dec. 27, 2007

(54) Method for Reversing Ventricular Dyssynchrony

(75) Inventor: Sanjeev Saksena, Green Brook, NJ (US)

Correspondence Address: HANSEN HUANG TECHNOLOGY LAW GROUP, LLP, 1725 EYE STREET, NW, SUITE 300, WASHINGTON, DC, 20006, US

(73) Assignee: EP MEDSYSTEMS, INC., West Berlin, NJ (US)

(21) Appl. No.: 11/426,704

(22) Filed: Jun. 27, 2006

(51) Int. Cl. A61N 1/365

(52) U.S. Cl. 607/18

(57) ABSTRACT

A method for reversing ventricular dyssynchrony uses intracardiac echocardiographic measured parameters to systematically determine an optimal, individualized configuration for a cardiac resynchronization stimulator device. This method is particularly relevant for patients with congestive heart failure. The algorithm evaluates improvement in aortic flow and in left ventricular ejection fraction as atrioventricular and interventricular delay parameters of the patient's resynchronization stimulator device are varied.

### FIELD OF THE INVENTION

This invention relates to medical diagnostic and therapeutic methods, and more particularly to methods for the treatment of cardiac sinus rhythm or atrial fibrillation.

### BACKGROUND OF THE INVENTION

The use of ventricular resynchronization therapy has been an important advance in the treatment of patients with heart failure. Disordered activation of the two lower chambers of the heart (interventricular dyssynchrony) has been identified as an important element in the deterioration of heart pump function and resulting cardiac failure. Implantable stimulator devices which separately deliver stimulation to the two chambers of the heart are frequently used treating this abnormality in what is referred to as cardiac resynchronization therapy.

The implantation of stimulator devices configured for cardiac resynchronization therapy is now routine clinical practice. However, 30-40% of patients receiving this therapy fail to achieve an adequate therapeutic response. It has been proposed that such failures of cardiac resynchronization therapy may be because patients are not appropriate candidates for the therapy or the therapy was not individualized to obtain an optimal outcome in a given patient. The current practice method is designed to optimize the use of cardiac resynchronization therapy in individual patients by evaluating their ventricular function during different program intervals delivered with the cardiac resynchronization device. Thus, there is a need for improved methods for treating interventricular dyssynchrony to improve the therapeutic response of many patients.

### SUMMARY OF THE INVENTION

The various embodiments of the present invention enable rapid and systematic optimization of electrical stimulation therapy delivered by an implantable cardiac resynchronization stimulator device in a patient with congestive heart failure using intracardiac echocardiographic measurements. The various embodiment methods are suitable for use in patients who are in normal sinus rhythm or in atrial fibrillation.

In overview, the various embodiments include the steps of advancing an intracardiac catheter with a phased array transducer into the right ventricle, positioning the phased array ultrasound transducer to view the left ventricle, measuring physiological characteristics of the heart using the phased array ultrasound transducer, saving the so-far optimal measurements and the parameters of the implantable cardiac resynchronization stimulator device producing them, reprogramming the implantable cardiac resynchronization stimulator device for each of various different atrioventricular intervals, repeating the above measuring and saving steps for each interval value, measuring physiological characteristics of the heart using the phased array ultrasound transducer, saving the so-far optimal measurements and the parameters of the implantable cardiac resynchronization stimulator device producing them, reprogramming the implantable cardiac resynchronization stimulator device for each of various interventricular delay times, repeating the above measuring and saving steps for each delay value, evaluating the optimized atrioventricular interval and interventricular delay, and analyzing the images for evidence of resynchronization.

### BRIEF DESCRIPTION OF THE DRAWINGS

The accompanying drawings, which are incorporated herein and constitute part of this specification, illustrate embodiments of the invention, and, together with the general description given above and the detailed description given below, serve to explain features of the invention.

FIG. 1 is a diagram of an intracardiac phased array ultrasound transducer positioned within the right ventricle of a (human) heart.

FIG. 2 is a functional system diagram of an ultrasound imaging system suitable for use in various embodiments.

FIG. 3 is a component system diagram of an ultrasound imaging system suitable for use in various embodiments.

FIG. 4 is a representation of a B-mode image of the left ventricle at diastole obtained by an intracardiac phased array ultrasound transducer positioned within the right ventricle.

FIG. 5 is a representation of a B-mode image of the left ventricle at systole obtained by an intracardiac phased array ultrasound transducer positioned within the right ventricle.

FIG. 6 is a representation of the left ventricle illustrating axes of measurement according to an embodiment.

FIG. 7 is a representation of the right ventricle illustrating axes of measurement according to an embodiment.

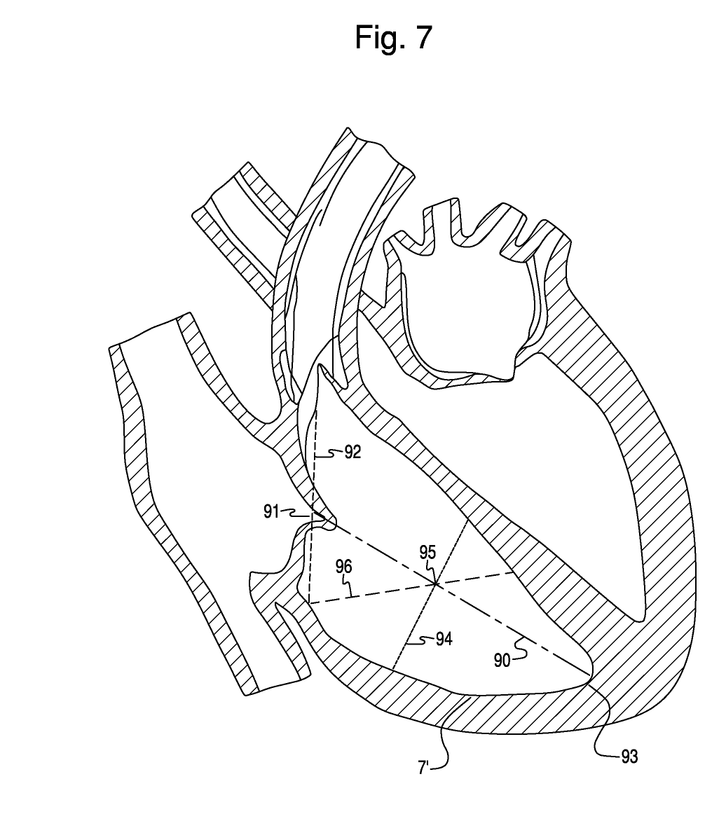

FIG. 8 is a representation of a ventricle including axes of measurements according to an embodiment.

FIG. 9 is a representation of a B-mode ultrasound image of the left ventricle at diastole with axes of measurement superimposed according to an embodiment.

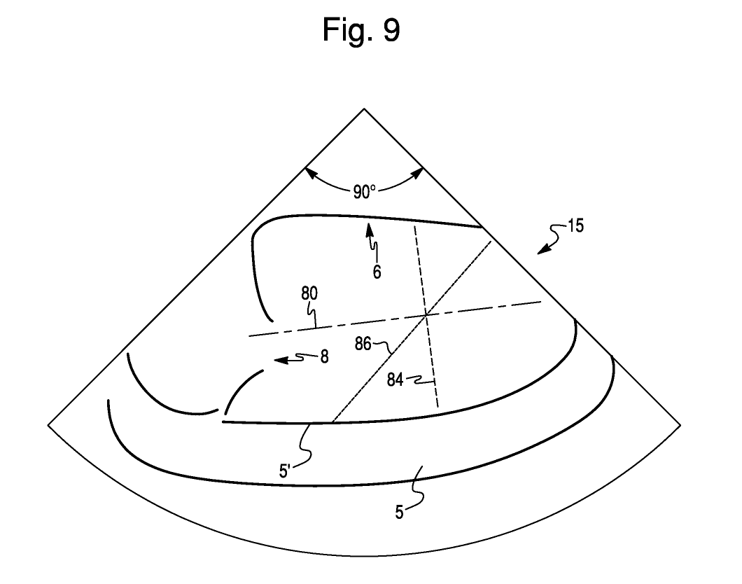

FIG. 10 is a flowchart of the steps of an embodiment of the present invention.

### DETAILED DESCRIPTION OF THE PREFERRED EMBODIMENTS

The various embodiments will be described in detail with reference to the accompanying drawings. Wherever possible, the same reference numbers will be used throughout the drawings to refer to the same or like parts.

As used herein, the terms “about” or “approximately” for any numerical values or ranges indicates a suitable dimensional tolerance that allows the part or collection of components to function for its intended purpose as described herein. Also, as used herein, the terms “patient”, “host” and “subject” refer to any human or animal subject and are not intended to limit the systems or methods to human use, although use of the subject invention in a human patient represents a preferred embodiment.

Phased array ultrasound imaging catheters are used for performing intracardiac echocardiography. Examples of phased array ultrasound imaging catheters and methods of using such devices in cardiac diagnosis are disclosed in U.S. Patent Application Publication Nos. 2004/0127798 to Dala-Krishna, et al., 2005/0228290 to Borovsky, et al., and 2005/0245822 to Dala-Krishna, et al., each of which is incorporated herein by reference in their entirety.

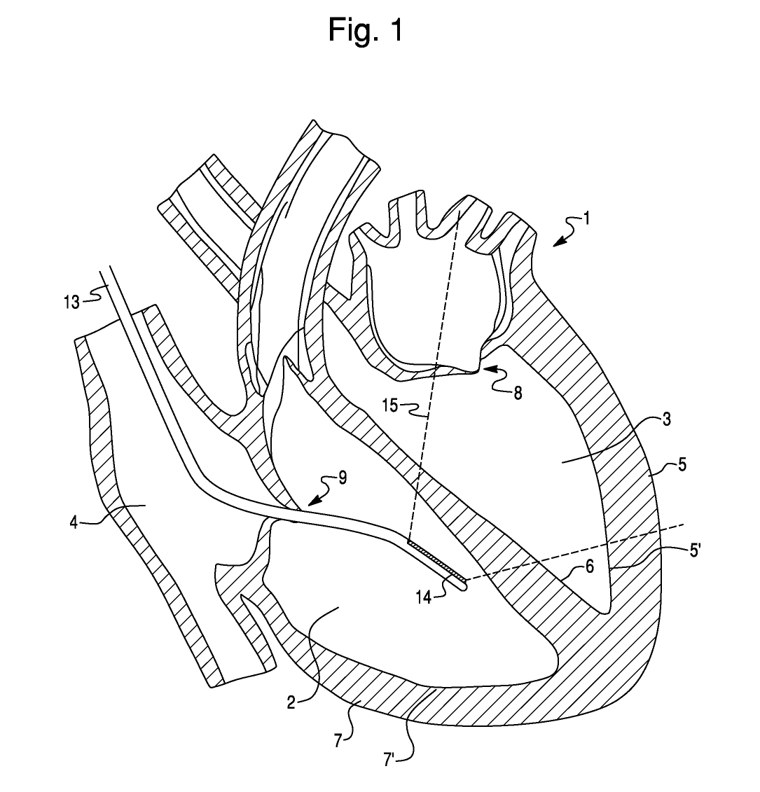

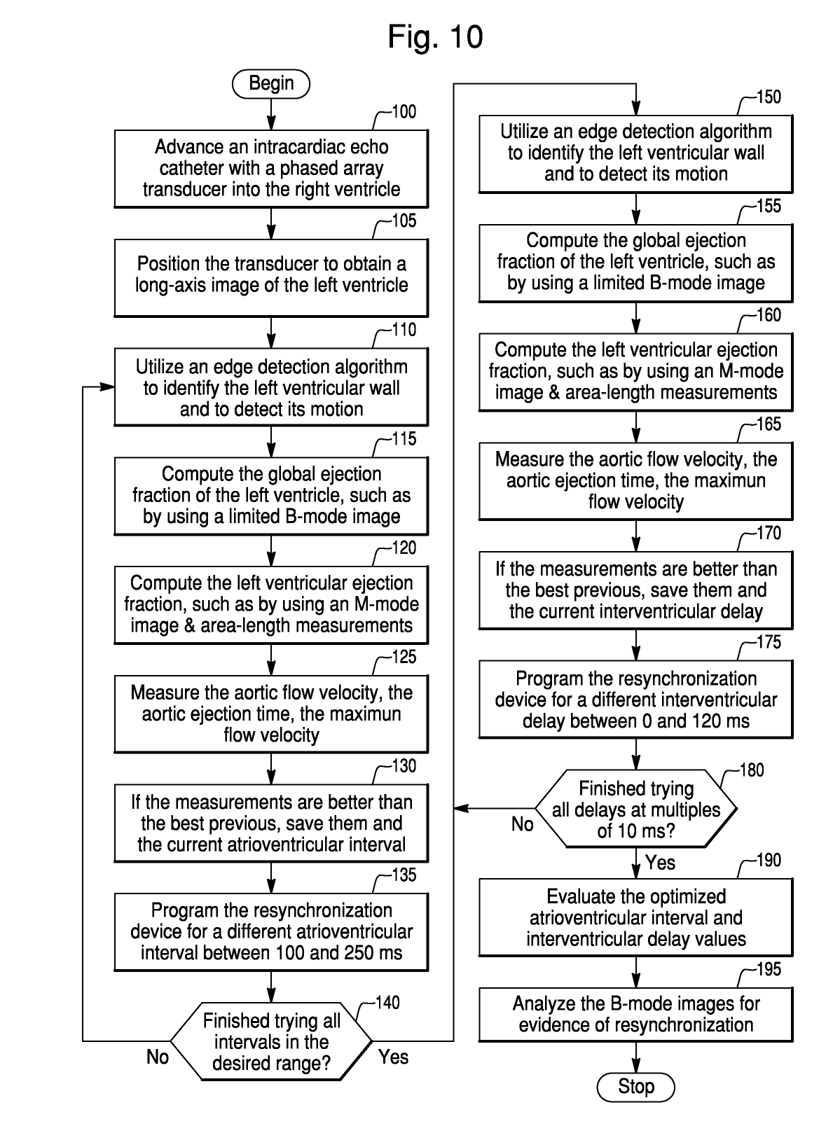

Referring to FIG. 1, an intracardiac echo catheter 10 with a phased array ultrasound transducer positioned near its tip 14 is advanced under fluoroscopic control into the right ventricle 2 of the heart 1. This is illustrated as step 100 in the flowchart of FIG. 10. As illustrated in FIG. 1, the transducer can positioned in the right ventricular 2 inflow tract in mid cavity in order to obtain a long axis view 15 of the left ventricle 3 (step 105 in FIG. 10). This allows imaging and evaluation of the left ventricular free wall 5 apex 8, base 8 and the septum 6. Procedures for positioning the phased array ultrasound transducer within the heart for imaging the left and right ventricles are described in U.S. patent application Ser. No. ______, entitled “Method For Evaluating Regional Ventricular Function And Incoordinate Ventricular Contraction” filed contemporaneous herewith and which is hereby incorporated by reference in its entirety.

Positioning of the intracardiac echo catheter 13 within the right ventricle may be accomplished before or after an implantable cardiac resynchronization stimulator device has been positioned in the patient with stimulator electrodes attached to the left and right ventricle walls. Typically, the intracardiac echo catheter 13 is used during the stimulator electrode attachment procedure since the imaging data can aid the practitioner in properly positioning the electrodes.

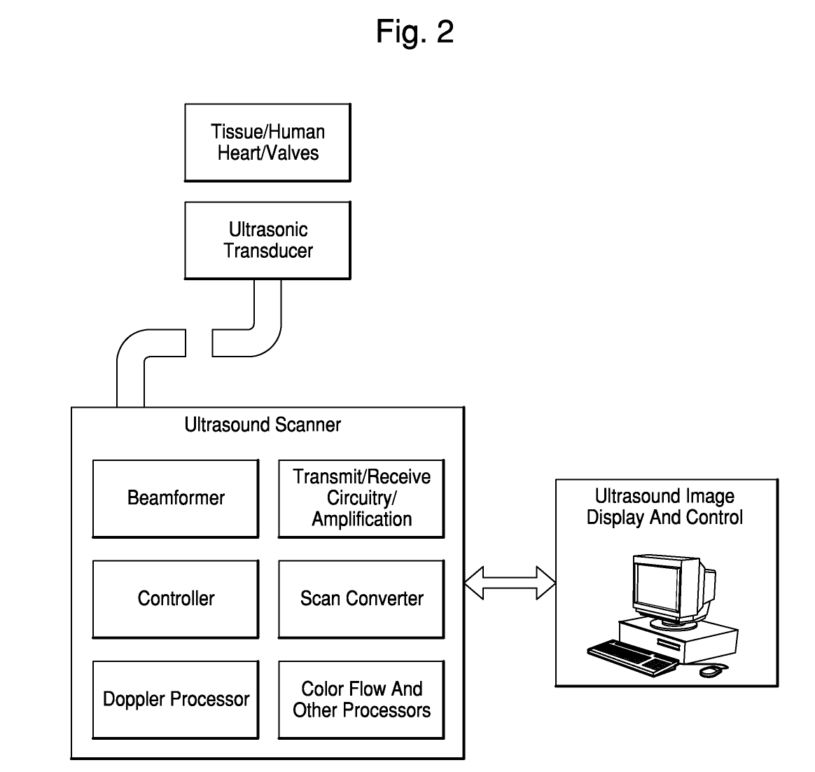

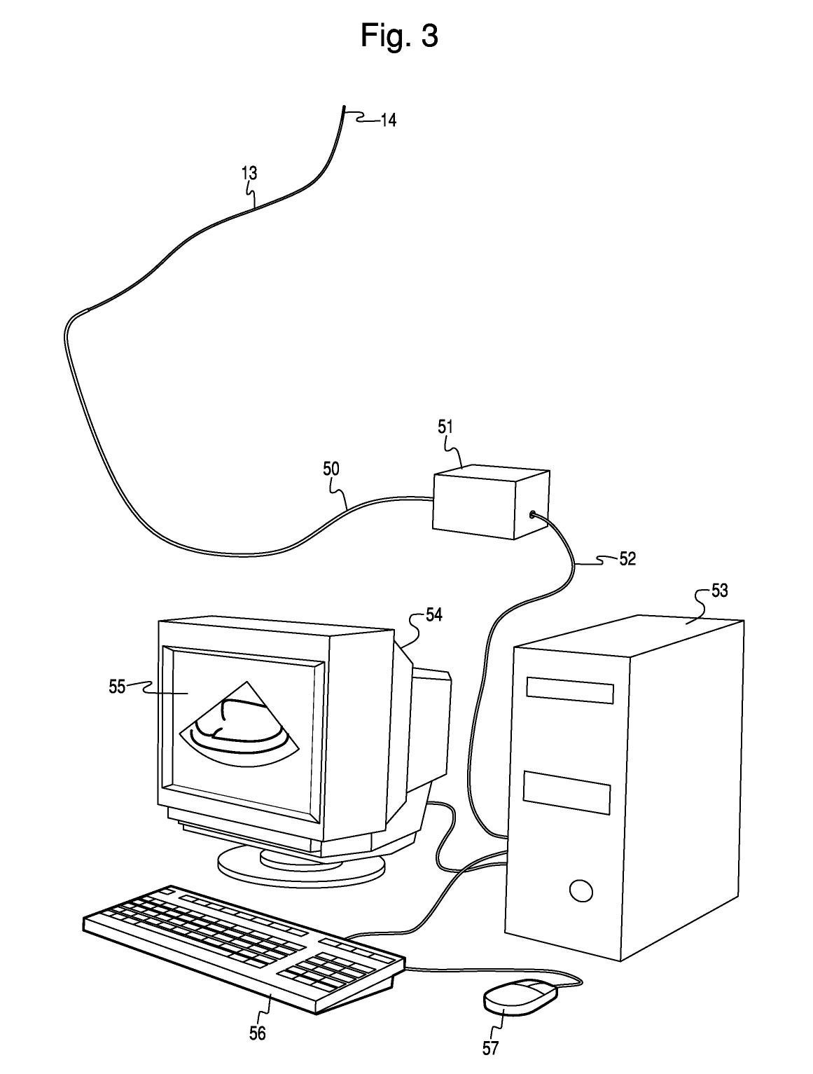

With the catheter phased array transducer 14 properly positioned within the heart, an ultrasound system, such as the ViewMate® Intracardiac Ultrasound Catheter System manufactured by EP MedSystems, Inc. of West Berlin, N.J., is connected to the catheter, an example of which is illustrated in FIGS. 2 and 3. The ultrasound system generates the electrical pulses which cause the transducer elements to emit ultrasound pulses. The ultrasound system also receives and processes the resulting echoes detected by the transducers. An ultrasound system includes a data cable 50 connected between the catheter 13 and an electrical isolation box 51. The data cable 50 may be connected to a handle (not shown) on the catheter 13 or may be an extension of the catheter itself. A data cable typically includes a number of coaxial cables, one for each phased array transducer element. The electrical isolation box 51 electrically isolates the catheter, thereby protecting the patient from stray currents that may be induced in the system or cabling 52 by radio frequency emissions and from fault currents that may result from an electrical short within the system equipment. An example of a suitable electrical isolation box 51 is described in U.S. patent application Ser. No. 10/997,898, published as U.S. Publication No. 2005-0124898 to Borovsky et al., and Ser. No. 10/998,039, published as U.S. Publication No. 2005-0124899 to Byrd et al., the entire contents of both of which are incorporated herein by reference in their entirety. Connected to the electrical isolation box 51, maybe another data cable 52 which conducts electrical information to a system processor 53. Coupled to the system processor 53 will typically be a monitor 54 for presenting a display 55 of the ultrasound data, and a keyboard 56 and pointing device 57 and/or other human interface device for accepting user commands and data inputs.

When the catheter is positioned within a patient's heart, the ultrasound system generates electrical pulses which cause the ultrasound transducers in the phased array transducer 14 to emit ultrasound pulses. By a controlling the phase lag of the pulses emitted by each transducer element within the phased array, a combined sound wave is generated with a preferential direction of propagation. Echoes from structures within the heart are received by the transducer elements and transformed into electrical pulses by the transducer. The electrical pulses are carried via the cables 50, 52 to the processor 53. The processor 53 analyzes the electrical pulses to calculate the distance and direction from which echoes were received based upon the time of arrival of the echoes received on each transducer element. In this manner, ultrasound energy can be directed in particular directions, such as scanned through a field of regard 15, and the resulting echoes interpreted to determine the direction and distance from the phased array that each echo represents.

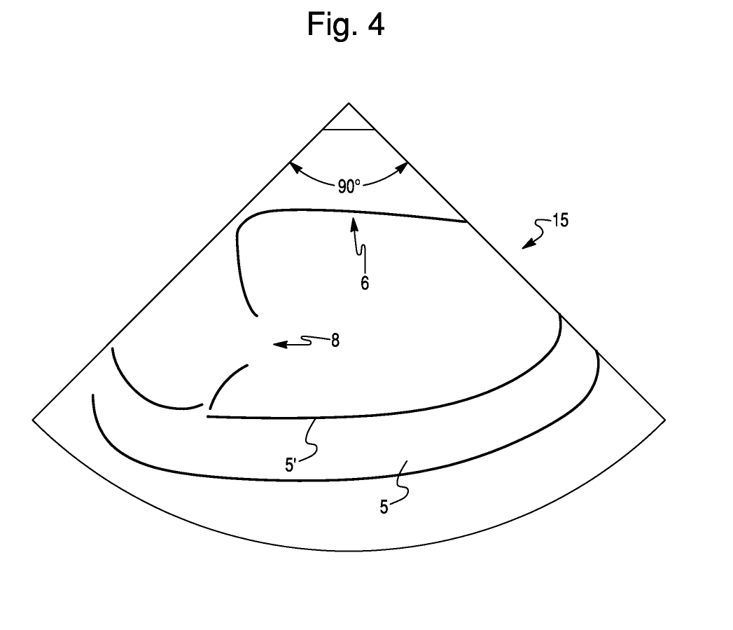

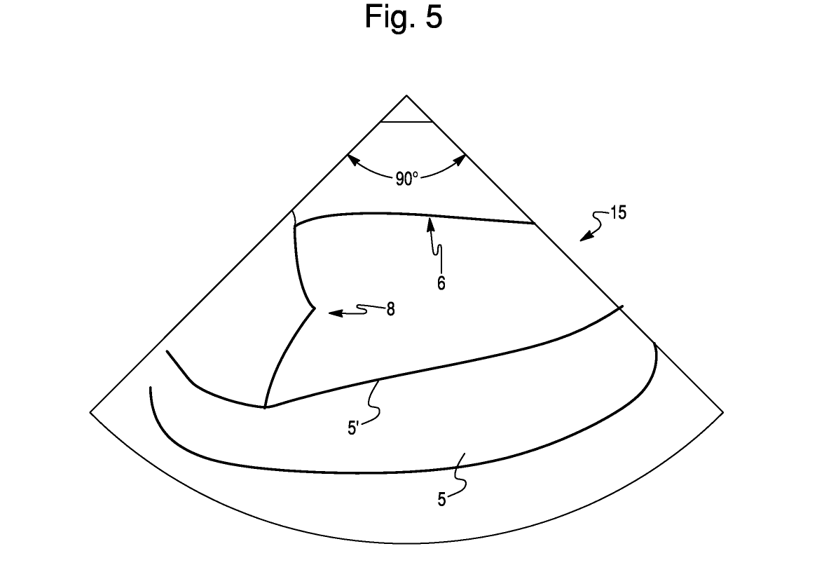

Scanning the ultrasound energy through a field of regard 15 generates a two-dimensional (2D) image of the heart, examples of which are shown in FIGS. 4 and 5. After a 2D scan is obtained, the catheter phased array transducer is rotated and another 2D image obtained, so that most of the endocardial surface of the ventricle (left or right) can be imaged. The B-mode ultrasound imaging technique is employed in this process. B-Mode ultrasound imaging displays an image representative of the relative echo strength received at the transducer. A 2-D image can be formed by processing and displaying the pulse-echo data acquired for each individual scan line across the angle of regard 15 of the phased array transducer. This process yields a two-dimensional B-mode image of the endocardial surface of the ventricle, examples of which is illustrated in FIGS. 4 and 5. Such images are obtained and recorded during approximately 10 or more cardiac cycles.

Since the scan rate of a phased array ultrasound transducer is much faster than the cardiac cycle, each scan presents a 2-D image at a particular time or phase in the cycle. Thus, individual scans, or a plurality of scans obtained at a particular phase or relative time within the cardiac cycle over a number of beats combined into an average image, can be used to provide a “freeze frame” image of the heart at particular instants within the cardiac cycle. Methods for combining and averaging multiple scans at a particular phase or relative time within the cardiac cycle (time gating) are described in U.S. application Ser. No. 11/002,661 published as U.S. Patent Publication No. 2005/0080336 to Byrd, et al., the entire contents of which are incorporated herein by reference in their entirety.

The freeze frame capability of B-mode images is used to obtain recordings particularly at the onset of QRS complex, which is near the end of diastole, and at the beginning of the T wave which is near the end of systole. FIG. 4 illustrates B-mode image of the left ventricle at diastole, and FIG. 5 illustrates a B-mode image of the left ventricle at systole. Sensing the QRS complex and T wave measurements obtained by electrocardiogram (ECG) sensors provides a signal that can be used to select a particular single image, or collect a number of images for averaging at the points of diastole and systole. The ECG sensors may be placed intracardiac via an electrode catheter or on the chest.

Automated edge-seeking algorithms or manual delineation of the endocardial signals is performed on the obtained images throughout the entire ventricle. Edge-seeking algorithms locate the edges of structure (e.g., ventricle walls) by noting a steep change in brightness (indicating echo intensity) from pixel to pixel. Alternatively, the cardiologist may define the edge of the endocardial surface 5′, 7′ in the image by manually tracing the edge using an interactive cursor (such as a trackball, light pen, mouse, or the like) as may be provided by the ultrasound imaging system. By identifying the edges of structure within an ultrasound image, an accurate outline of ventricle walls can be obtained and other image data ignored. The result of this analysis is a set of images and dimensional measurements defining the position of the ventricle walls at the particular instants within the cardiac cycle at which the “freeze frame” images were obtained. The dimensional measurements defining the interior surface 5′ or 7′ of the endocardium can be stored in memory of the ultrasound system and analyzed using geometric algorithms to determine the volume of the ventricle.

Edge detection algorithms applied in the ultrasound system to the ultrasound echo image data to identify the endocardial surface of the left ventricular wall 5 can generate an image of the ventricle structures such as illustrated in FIGS. 4 and 5. By identifying the ventricular wall 5 structure, the system is able to detect and measure wall motion (step 110 in FIG. 10). A B-mode image, illustrated in FIG. 4, of the left ventricle from the phased array ultrasound transducer is used to measure global ejection fraction of the heart (step 115) using methods such as described herein and in co-pending U.S. patent application Ser. No. ______ already incorporated by reference. An M-mode image of the left ventricle from the phased array ultrasound transducer is used to measure the length and area of the left ventricle. Then these measurement results are used to estimate the left ventricular ejection fraction (step 120) using the following estimation methods.

For the left ventricle 3, an image of most of if not the entire endocardium can be obtained, preferably from the base of the aortic valve to the left ventricular apex and across back to the base of the aortic valve. An illustration of such an ultrasound image at diastole is provided in FIG. 4. The aortic valve plane is imaged and defined using edge-seeking algorithms to complete the delineation of the cavity enclosing the blood flow. In particular, these images are obtained for the end-diastolic and end-systolic portions of the cardiac cycle, FIGS. 4, 5, thereby measuring the dimensions and contours of the ventricle walls at the instances of maximum (FIG. 4) and minimum volume (FIG. 5).

Having obtained dimensional measurements of the left ventricle 3 from the ultrasound images at or near diastole and systole, the ultrasound system processor can calculate the volume in the ventricle at both instances and, from the ratio of these two volumes, calculate the ejection ratio of the left ventricle 3.

While FIGS. 4 and 5 and the foregoing description address the left ventricle 3, similar images are obtained for the right ventricle 2, except that the image extends from the base of the tricuspid value 9 to the right ventricular apex 93 and across back to the base of the tricuspid value 9. From the images of the right ventricle 2, similar calculations of ventricle volume are obtained at points in the cardiac cycle of maximum and minimum volume to calculate the ejection fraction of the right ventricle 2.

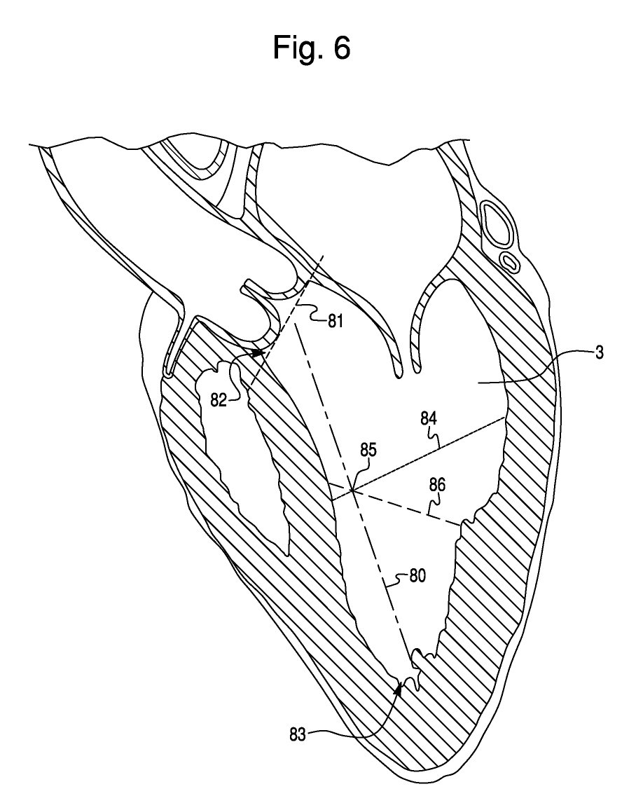

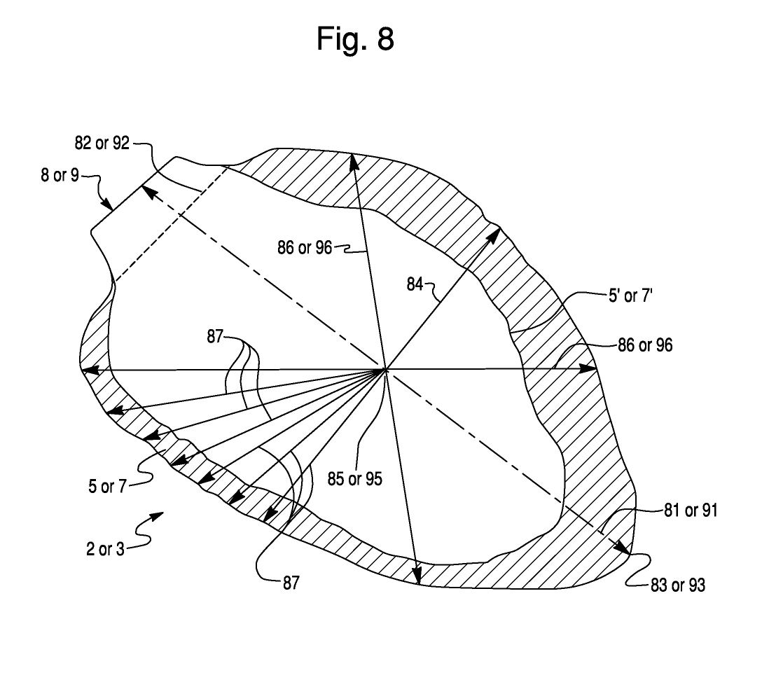

Ventricle ejection fraction can be estimated based on linear dimensional measurements of the ventricle without calculating the volume of the ventricle. In this embodiment, the long axis 80 of the left ventricle 3 is defined from the mid point 81 of the aortic valve plane 82 to the left ventricular apex 83, as illustrated in FIG. 6. Similarly, the long axis 90 of the right ventricle 2 is defined from the mid plane 91 of the tricuspid of the pulmonic valve plane 92 to the right ventricular apex 93. The long axis 80, 90 from the midpoint of the valvular plane to the apex is then subtended and bisected. The perpendicular axis 84, 94 at the midpoint 85, 95 of the long axis 80, 90 is used for subtending the short axis at a perpendicular. Additional radians 86, 96 are then subtended at an acute angle, such as 30 or 45 degree angles, from the central point 85, 95 of the ventricle as defined by the intersection of the two axes. These radial axes are superimposed along with the short and long axes on the end-systolic and end-diastolic frames of the ventricle B-mode image, as illustrated in FIG. 8 for the left ventricle.

The area in each segment as defined by the radial axes is then planimetered and automatically computed. The area in each sector of the ventricle or the fractional shortening along the radian in the sector can be used as a measure of regional ventricular function and ejection fraction. The difference in area between the measured area in the end-diastolic image and the measured area in the end-systolic image characterizes the regional ejection fraction for the region of the heart subtended by each such pair of corresponding sectors. This change in area of a region may be used to estimate the regional ejection fraction for the measured segment. This estimate is based upon the assumption that the length of the long axis 80, 90 does not change significantly during contraction, so that the change in volume is proportional to the change in area of a transverse cross section. In this manner, the regional ejection fraction for each of the segments can be easily calculated by the ultrasound system processor to provide ejection fractions for multiple regions of the ventricle.

The definition of axes and radians is further illustrated in FIG. 8 which shows a stylized ventricle which may be either the left ventricle 3 or right ventricle 2. Referring to FIG. 8, an embodiment method defines a long axis 90 to extend from the midplane of the tricuspid 9 of the pulmonic valve plane to the right ventricular apex 93. For the left ventricular cavity 3, the method defines the long axis 80 to extend from the mid point 91 of the aortic valve plane 91 to the left ventricular apex 83. The long axis 80, 90 from the midpoint 81, 91 of the valvular plane 82, 92 to the apex 83, 93 contains a midpoint 85, 95, which bisects the long axis 80, 90. A transverse line or plane 84, 94 is defined at the midpoint perpendicular to the long axis 80, 90. Radials 86, 96 are then defined in the plane of the cross-sectional image at an acute angle to the transverse axis 84, 94 and crossing the midpoint 81, 91. The ultrasound system processor may construct further radials 87 extending from the midpoint 85, 95 of the long axis 80, 90 at a plurality of angles (e.g., multiples of 30 or 45 degrees) with respect to the long axis 80, 90. Each radial 87 terminates where it intersects the endocardial wall 5′ or 7′ in the ultrasound image. Each half of the long axis 80, 90 also forms a radial.

The embodiment method may approximate the area of each sector or region in an image of the ventricular cavity 2 or 3 being examined as the sum of the areas of multiple, small, disjoint, abutting triangles which effectively subdivide and cover the sector or region. For example, each triangle may have the long axis bisection point 85, 95 as one vertex, and two sides defined by radials 87 from the bisecting midpoint 85, 95 terminating at the edge of the endocardial wall 5′ or 7′.

As an alternative or addition to the area method of estimating ejection fraction, the change in length of each of the radials 84, 86, 87 can provide information characterizing the instantaneous ejection fraction by monitoring the endocardial wall motion in the direction along each radial. These radials 84, 86, 87 relate to specific anatomic regions of the imaged heart ventricle. The values and relative timing of the regional ejection fractions, which correspond to the various radials 84, 86, 87, can be used to assess the effect of alternative interventions as described herein.

Calculation of regional ejection fractions can also be accomplished at various predefined points in the systolic cycle, such as, for example, at or near early (˜33%), mid (˜50%), late (˜67%) and end (˜100%) points of the systolic period of ventricular contraction. This can be accomplished by subtracting the area of each segment at the predefined point in the cycle from the area of the segment measured at diastole.

Overall global ejection fraction can be estimated by summing all of the regional ejection fractions obtained according to the above methods. The global ejection fraction can be measured at different predefined points in the systolic cycle, such as at or near early (˜33%), mid (˜50%), late (˜67%) and end (˜100%) points of the systolic period of ventricular contraction. This calculation permits evaluation of ventricular ejection fraction at different points in the cardiac cycle. By calculating the ventricular ejection fraction at different points in the cardiac cycle, detection and evaluation of ventricular dysynchronous contraction is possible.

The foregoing measurements and estimations of regional and global ventricle ejection fraction can be performed in sinus rhythm or atrial fibrillation prior to applying resynchronization stimulation in order to document the baseline state of an individual patient.

Once a baseline state of the patient's heart function has been obtained according to the methods described above, the practitioner can use spectral Doppler ultrasound to measure the aortic flow velocity, the time duration of the aortic ejection, and its maximum velocity (step 125). Methods for measuring aortic flow velocity, the time duration of the aortic ejection and maximum velocity are obtained by measuring the Doppler shift of the ultrasound echoes as is well known in the cardiac ultrasound imaging practice. Other measurements, such as an estimate of the volume of blood ejected, can be used instead of or in addition to these measurements. Spectral Doppler ultrasound measurements are also obtained in sinus rhythm or atrial fibrillation prior to applying resynchronization stimulation in order to document the baseline state in an individual patient.

B-mode and M-mode measurements of left ventricular ejection fraction, maximum aortic flow velocity, overall aortic flow with an area computation and an aortic ejection time provide the practitioner with information useful for setting the current timing configuration of the patient's cardiac resynchronization stimulator device. The resynchronization stimulator device configuration parameters include at least the atrioventricular interval and the interventricular delay timing.

Using the atrioventricular interval and the interventricular delay timing settings obtained from the patient's baseline measurements, the practitioner initially programs the implantable resynchronization stimulator device and initiates stimulator operation.

With stimulator operation initiated, the above measurement steps, beginning with the application of the endocardial surface edge detection, are repeated. The measurement steps provide measurements of ventricle dimensions which are used to estimate ventricle ejection fraction which is indicative of the heart's function with the initial resynchronization stimulator device settings. In particular, measurements and estimations indicative of the heart's function include one or more of the left ventricular ejection fraction, maximum aortic flow velocity, overall aortic flow with an area computation and an aortic ejection time.

The practitioner then adjusts the programmed atrioventricular interval parameter values to a new setting or settings (step 135) and the measurements are repeated. The measurement steps provide measurements of the heart's left ventricular ejection fraction, maximum aortic flow velocity, overall aortic flow with an area computation and an aortic ejection time with the new resynchronization stimulator device settings. When each set of measurements is obtained, the practitioner again adjusts the programmed atrioventricular interval parameter values to new settings (repeating step 135) and repeats the measurement steps to obtain ventricular ejection fraction, maximum aortic flow velocity, overall aortic flow with an area computation and aortic ejection time values. By incrementally adjusting settings and repeating this process, the measures of ventricle function can be acquired at across a range of atrioventricular interval parameter settings. In performing this sequence, the practitioner adjust the atrioventricular intervals in increments of between about 5 and about 10 milliseconds to cover the range of settings. The range of atrioventricular interval settings may be between about 100 milliseconds and about 250 milliseconds.

As the above steps are repeated, but before a new atrioventricular interval parameter is set, the system or practitioner notes which device configuration produces the maximum aortic flow and the best left ventricular ejection fraction so far (step 130), as well as noting the measurements produced thereby.

When the optimal heart efficiency measurements over the full range of atrioventricular intervals have been obtained, or when it is clear that no better measurements will been obtained, the adjust-measure-repeat cycle is ended (step 140), and the atrioventricular interval which produced the optimal measurements (the optimal atrioventricular interval) is stored in memory along with the final optimal left ventricular ejection fraction, maximum aortic flow velocity, overall aortic flow with an area computation and an aortic ejection time measurements (memorized by step 130).

After determining the optimal atrioventricular interval, interventricular conduction delay is then optimized as follows. With the atrioventricular interval setting of the implantable cardiac resynchronization stimulator device fixed at the optimal atrioventricular interval, the flow and ejection fraction measurements described above are repeated with the stimulator device settings adjusted for each set of measurements to an interventricular delay in a sequence of interventricular delays ranging preferably from about 0 to about 120 milliseconds in increments of about 5 to about 10 milliseconds (step 175). Additionally, measurements are taken with the interventricular delay set so the left ventricle precedes the right ventricle and/or so the right ventricle precedes the left ventricle. At each delay value in the range, the specific delay is associated with the flow and ejection measurement values, such as noted by the practitioner or stored in memory as a linked data set.

As the flow and ejection measurements are taken at each interval setting of the interventricular delay (note that steps 150 through 165 repeat the measurements taken in steps 110 through 125), the optimal flow and ejection measurements so far and the associated delay parameter are retained (step 170). When the measurements over the full range of intervals have been obtained, or when it is clear that no better measurements will be obtained, the adjust-measure-repeat cycle is ended (step 180) and the interventricular delay which produced the optimal measurements (the optimal interventricular delay) is retained along with the final optimal measurements (as memorized in step 170).

The final retained atrioventricular and interventricular delay parameters are set in the resynchronization stimulator device, and a set of measurements are conducted to determine the percent increase in aortic flow and the percent improvement in the left ventricular ejection fraction achieved compared to the baseline measurements (step 190).

Finally, B-mode ultrasound images are analyzed for evidence of actual resynchronization of the left and right ventricles under stimulation by the device (step 195). This resynchronization is measured by comparing the timing delay of movements within the septum on the posterior wall.

In pilot studies, improvement in left ventricle ejection fraction of greater than 10% was observed following use of this method, with virtually each patient showing improvement. Such outcomes represent substantial improvement in therapeutic results over current experience where 30-40% of patients fail to show improvement with cardiac resynchronization devices.

It should be noted that there are other embodiments or improvements that would be obvious to those familiar with the field of this invention. For example, the order of the two parametric optimizations (atrioventricular and interventricular parameters) may be reversed. Also, the optimizations can be iterated and interleaved, which will allow detecting interdependencies (such as false maximums) between atrioventricular and interventricular delay values and refining both atrioventricular and interventricular delay values together. Also, steps of the method may be performed in a different order than illustrated in FIG. 10, such as reversing steps 120 and 125.

While the present invention has been disclosed with reference to certain preferred embodiments, numerous modifications, alterations, and changes to the described embodiments are possible without departing from the sphere and scope of the present invention, as defined in the appended claims. Accordingly, it is intended that the present invention not be limited to the described embodiments, but that it have the full scope defined by the language of the following claims, and equivalents thereof.

I claim

1. A method for configuring a ventricular resynchronization stimulator device for the heart of an individual patient, comprising:
measuring at least one of the heart's left ventricular ejection fraction, maximum aortic flow velocity, and overall aortic flow with the ventricular resynchronization stimulator device programmed at incremental atrioventricular interval delay settings within a range of atrioventricular interval delay settings;
identifying the incremental atrioventricular interval setting that results in an optimal measurement of at least one of the heart's left ventricular ejection fraction, maximum aortic flow velocity, and overall aortic flow;
measuring at least one of the heart's left ventricular ejection fraction, maximum aortic flow velocity, and overall aortic flow with the ventricular resynchronization stimulator device programmed at incremental interventricular interval delay settings within a range of interventricular interval delay settings; and
identifying the incremental interventricular interval setting that results in an optimal measurement of at least one of the heart's left ventricular ejection fraction, maximum aortic flow velocity, and overall aortic flow.

2. The method according to claim 1, further comprising:
positioning an intracardiac echo catheter in the patient's right ventricular inflow tract in mid cavity to obtain a long axis view of the left ventricle of the patient's heart; and
identifying the endocardial surface of the left ventricular wall and detecting motion of the left ventricular wall.

3. The method according to claim 2, wherein computing the global ejection fraction is performed using a limited B-mode image of the left ventricle.

4. The method according to claim 2, wherein computing the left ventricular ejection fraction is performed using an M-mode image of the left ventricle.

5. The method according to claim 1, wherein the range of atrioventricular interval delay settings is between about 100 milliseconds and about 250 milliseconds.

6. The method according to claim 1, wherein the incremental atrioventricular interval delay settings are separated by between about 5 milliseconds to about 10 milliseconds.

7. The method according to claim 1, wherein the range of interventricular delay settings is between about 0 milliseconds and 120 milliseconds with the left ventricle preceding the right ventricle.

8. The method according to claim 1, wherein the range of interventricular delay settings is between about 0 milliseconds and about 120 milliseconds with the right ventricle preceding the left ventricle.

9. The method according to claim 1, wherein the incremental interventricular delay settings are separated by about 5 milliseconds to about 10 milliseconds each.

10. A method for configuring a ventricular resynchronization stimulator device for the heart of an individual patient, comprising:
measuring at least one parameter indicative of the heart's function with the ventricular resynchronization stimulator device programmed at each of a plurality of atrioventricular interval delay settings within a range of atrioventricular interval delay settings;
identifying one of the plurality of atrioventricular interval delay settings that results in an optimal measurement of the heart's function;
measuring at least one parameter indicative of the heart's function with the ventricular resynchronization stimulator device programmed at each of a plurality of interventricular interval delay settings within a range of interventricular interval delay settings; and
identifying one of the plurality of interventricular interval delay setting that results in an optimal measurement of the heart's function.

11. The method according to claim 10, wherein the at least one parameter indicative of the heart's function is one or more global ejection fraction, ventricular ejection fraction, aortic flow velocity, aortic ejection time, and maximum flow velocity.

12. The method according to claim 11, wherein the at least one parameter indicative of the heart's function is measured using an phased array ultrasound imaging catheter positioned in the patient's right ventricular inflow tract in mid cavity to obtain a long axis view of the left ventricle of the patient's heart.

13. The method according to claim 10, wherein the range of atrioventricular interval delay settings is between about 100 milliseconds and about 250 milliseconds.

14. The method according to claim 10, wherein the plurality of atrioventricular interval delay settings are separated by between about 5 milliseconds to about 10 milliseconds.

15. The method according to claim 10, wherein the range of interventricular delay settings is between about 0 milliseconds and 120 milliseconds with the left ventricle preceding the right ventricle.

16. The method according to claim 10, wherein the range of interventricular delay settings is between about 0 milliseconds and about 120 milliseconds with the right ventricle preceding the left ventricle.

17. The method according to claim 10, wherein the plurality of interventricular delay settings are separated by about 5 milliseconds to about 10 milliseconds each.

18. The method according to claim 12, further comprising programming the ventricular resynchronization stimulator device with the identified one of the plurality of atrioventricular interval delay settings and the identified one of the plurality of interventricular interval delay settings that results in an optimal measurement of the heart's function;

19. The method according to claim 18, further comprising observing the heart using the phased array ultrasound imaging catheter for the evidence of resynchronization indicated by a timing delay within the septum on the posterior wall.

20. The method according to claim 10, further comprising setting initial parameters of the resynchronization stimulator device based upon baseline measurements of the heart's global ejection fraction and left ventricular ejection fraction.

21. The method according to claim 10, wherein the interventricular interval delay settings show only improved left ventricular function with only stimulation of the right or left ventricle.
